(19) United States
(12) Patent Application Publication
(10) Pub. No.: US 2020/0237948 A1
(43) Pub. Date: Jul. 30, 2020

(54) AIR FRESHENER DISPENSER

(71) Applicant: Battelle Memorial Institute, Columbus, OH (US)

(72) Inventors: Matthew Vaughan, Columbus, OH (US); Brett Burton, Columbus, OH (US); David Bartholomew, Columbus, OH (US); Trevor Tallos, Columbus, OH (US); David Chase, Columbus, OH (US); Bryan D. Balaban, Groove City, OH (US); Jackson Brengman, Lancaster, OH (US)

(21) Appl. No.: 16/774,702

(22) Filed: Jan. 28, 2020

Related U.S. Application Data
(60) Provisional application No. 62/797,553, filed on Jan. 28, 2019.

(51) Int. Cl. A61L 9/03; A61L 9/12; A61L 9/14

(52) U.S. Cl. CPC A61L 9/03; A61L 9/12; A61L 2209/133; A61L 2209/12; A61L 2209/111; A61L 9/14

(57) ABSTRACT

An air freshener includes an electrically powered fragrance dispenser having a connector configured to connect with a fragrance container. A light source transmits a light beam into the fragrance container at a non-normal angle of incidence respective to a wall of the fragrance container, and a photodetector is positioned in the path of one of (i) the light beam after passing through the fragrance container when the light beam is not refracted by fragrance liquid in the fragrance container and (ii) the light beam after passing through the fragrance container when the light beam is refracted by fragrance liquid in the fragrance container. In another approach, the sensor for detecting empty includes a vibrator and a vibration sensor, and an electronic processor is programmed to determine whether the fragrance container is empty of fragrance liquid based on the detected vibration of the fragrance container.

This application claims the benefit of U.S. Provisional Application No. 62/797,553 filed Jan. 28, 2019. U.S. Provisional Application No. 62/797,553 filed Jan. 28, 2019 is incorporated herein by reference in its entirety.

### BACKGROUND

The following relates to the air freshener arts, fluid dispenser arts, and fluid dispenser monitoring arts, and related arts.

Air fresheners are commonplace in both residences and commercial spaces. The fragrance dispersed by the air freshener is typically supplied in a liquid form (often with substantial viscosity), e.g. in a bulb or other container. As a non-limiting example, the fragrance may be disposed in a Dowanol™ hydrophilic glycol ether fluid. The bulb is designed to connect with a dispenser which disperses the fragrance using a nebulizer, mister, vaporizer, or other dispersal technology. Known dispensers operate using various technologies, e.g. thermal, piezoelectric, propellent-driven, nebulizer or so forth. The air freshener dispenser is usually electrically powered, most conveniently by way of house electricity (e.g., via a cord terminating in a standard AC power plug, or alternatively via an AC power plug built into the air freshener dispenser unit) although battery powered air fresheners are also known.

The fluid containing the fragrance eventually runs out, at which point the bulb or other fragrance container must be refilled (if the bulb is refillable) or replaced (if the bulb is a disposable consumable). In practice, however, it can be difficult to ensure such replacement is done in a timely manner. By design, an air freshener operates without user interaction, so that it is easy to forget to check whether it is empty. Also, while the bulb is typically transparent or translucent so that the fluid level can be visually observed, the air freshener is sometimes placed in an inconspicuous or hidden location, such as being plugged into an electrical outlet located behind a chair or other furniture. In a commercial setting, a further difficulty is that maintenance personnel may only check the air freshener at occasional intervals, which can result in the air freshener being out of fragrance for an extended time period.

While air fresheners are a typical product to which aspects disclosed herein pertain, it will be appreciated that similar issues relating to timely refill of a consumable fluid arise in other residential and commercial dispensers, such as hand sanitizers.

### BRIEF SUMMARY

In accordance with some illustrative embodiments disclosed herein, an air freshener comprises: an electrically powered fragrance dispenser having a connector configured to connect with a fragrance container which is transparent or translucent, wherein the electrically powered fragrance dispenser is operative to generate a fragrance output wherein the generation of the fragrance output consumes a fragrance liquid stored in the fragrance container; a light source disposed on or in the electrically powered fragrance dispenser and positioned to transmit a light beam into the fragrance container at a non-normal angle of incidence respective to a wall of the fragrance container upon which the light beam impinges; and a photodetector disposed on or in the electrically powered fragrance dispenser and at a position which is in the path of one of (i) the light beam after passing through the fragrance container when the light beam is not refracted by fragrance liquid in the fragrance container and (ii) the light beam after passing through the fragrance container when the light beam is refracted by fragrance liquid in the fragrance container.

In accordance with some illustrative embodiments disclosed herein, a method is disclosed of monitoring whether a liquid container connected with a fluid dispenser is empty. The fluid dispenser is operated to dispense fluid by consuming liquid contained in the liquid container. A light beam is directed into the liquid container. It is detected whether the light beam passes through a chord of a cross-section of the liquid container. An indication is output, via a visual indicator or a wireless transmitter or transceiver, that the fluid dispenser should be refilled or replaced if the light beam is one of (i) detected to pass through the chord of the cross-section of the liquid container or (ii) not detected to pass through the chord of the cross-section of the liquid container.

In accordance with some illustrative embodiments disclosed herein, an air freshener comprises: an electrically powered fragrance dispenser having a connector configured to connect with a fragrance container which is transparent or translucent, wherein the electrically powered fragrance dispenser is operative to generate a fragrance output wherein the generation of the fragrance output consumes a fragrance liquid stored in the fragrance container; a vibrator disposed on or in the electrically powered fragrance dispenser and positioned to induce a vibration of the fragrance container; a vibration sensor disposed on or in the electrically powered fragrance dispenser and positioned to detect the vibration of the fragrance container; and an electronic processor programmed to determine whether the fragrance container is empty of fragrance liquid based on a frequency and/or amplitude of the detected vibration of the fragrance container.

### BRIEF DESCRIPTION OF THE DRAWINGS

FIGS. 1, 2, and 3 diagrammatically illustrate perspective, side, and front views, respectively, of an air freshener.

FIG. 4 diagrammatically shows Section S-S indicated in FIG. 3 according to a first embodiment.

FIG. 5 diagrammatically plots the photodiode voltage of the photodiode of the embodiment of FIG. 4 as a function of fluid level in the fluid bulb.

FIGS. 6, 7, and 8 diagrammatically show a mobile device running an application program (“app”) monitoring the air freshener of FIGS. 1-3, where: FIG. 6 shows a suitable display when the fluid bulb is not empty; FIG. 7 shows a suitable display when the fluid bulb is empty; and FIG. 8 shows a suitable display when the fluid bulb is not installed.

FIG. 9 diagrammatically shows infrared mapping of the impact of the fragrance container on the beam shape.

FIG. 10 diagrammatically shows Section S-S indicated in FIG. 3 according to a second embodiment.

### DETAILED DESCRIPTION

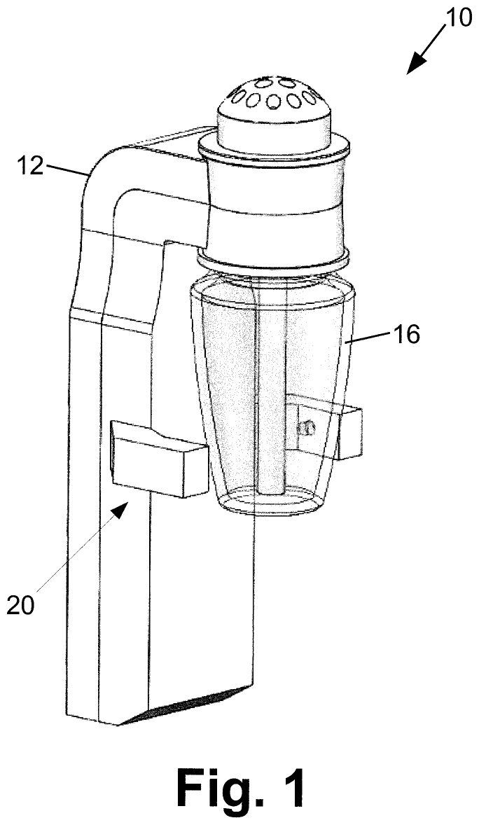

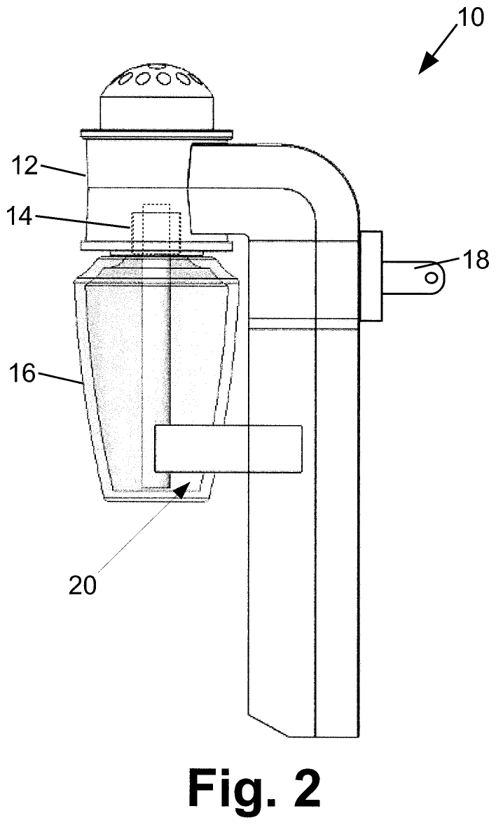

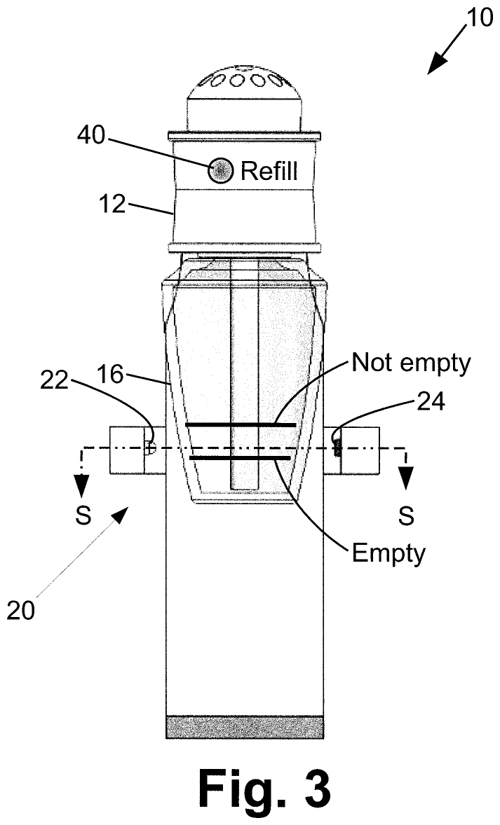

With reference to FIGS. 1-3, perspective (FIG. 1), side (FIG. 2), and front (FIG. 3) views, respectively, of an air freshener 10 are shown. The air freshener 10 includes an electrically powered fragrance dispenser 12 having a connector 14 (internal component diagrammatically indicated in phantom only in FIG. 2) configured to connect with a fragrance container 16. The fragrance container is transparent or translucent, as is typical as this allows the user to visually see the level of fragrance liquid remaining in the container 16. In some embodiments, the connector 14 of the electrically powered fragrance dispenser 12 is a threaded connector whereby the fragrance container 16 screws onto the electrically powered fragrance dispenser 12. However, this is merely an illustrative example, and other types of connectors are contemplated; as another non-limiting illustrative example, the connector 14 may be a bayonet connector with a cylindrical male side (on one of the dispenser 12 or container 16) with radial pins, and a female receptor (on the other of the dispenser 12 or container 16) with L-shaped slots mating with the radial pins and optionally with springs to keep the two parts locked together.

The electrically powered fragrance dispenser 12 receives electrical power from an external source, for example via a two-prong AC power plug 18 (shown only in FIG. 2; or, alternatively, three-prong plug with a third ground pin, the detailed shape of the two- or three-prong plug may be chosen to comport with regional standards, e.g. U.S. versus European-style AC power plugs). It is also alternatively contemplated for the electrically powered fragrance dispenser 12 to receive electrical power from an on-board battery or batteries (not shown), or to have a 12 VDC connector for use in a vehicle, and/or so forth. The illustrative AC power plug 18 is built into the fragrance dispenser 12 so that it may be used to mount the fragrance dispenser 12 to a wall via an electrical wall plug receptacle; however, in other embodiments the fragrance dispenser may be a tabletop unit or otherwise supported or mounted, and the electrical power delivered via an electrical cord terminating in a power plug (or, alternatively, via internal battery or batteries as mentioned previously).

The electrically powered fragrance dispenser 12 is operative to generate a fragrance output, for example by thermal heating of fragrance liquid drawn from the fragrance container 16 via the connector 14, or by piezoelectric energizing of the fragrance liquid drawn from the fragrance container 16 via the connector 14, or by propellent-driven output of the fragrance liquid, or nebulization of the fragrance liquid, and/or so forth. The fragrance output is typically in the form of a mist, vapor, or other airborne fragrance output. The electrically powered fragrance dispenser 12 includes the mechanical hardware for generating the airborne fragrance output (e.g. the nebulizer hardware, the pump providing propellant, the piezoelectric hardware, the heater or so forth) and also the housing containing/supporting this hardware. For example, in the illustrative example of FIGS. 1-3 the electrically powered fragrance dispenser 12 includes the generally “L”-shaped housing which provides structural support for mounting to the wall via the AC electrical plug 18 and defines a partial recess within which the installed fragrance container 16 is located.

The generation of the airborne fragrance output by the electrically powered fragrance dispenser 12 consumes fragrance liquid stored in the fragrance container 16. Typically, the fragrance liquid is a solution or suspension of fragrance molecules, particles, or the like which are dissolved, suspended, or otherwise disposed in a solvent or other host liquid. In one non-limiting example, the host liquid containing the fragrance is a Dowanol™ hydrophilic glycol ether fluid; however, other host liquids such as water are additionally or alternatively contemplated.

With continuing reference to FIGS. 1-3, the disclosed air freshener 10 advantageously includes a sensor assembly 20 for detecting whether the fragrance container 16 is empty. The term “empty” as used herein denotes the amount or level of fragrance liquid in the fragrance container 16 is low enough to cause the sensor assembly 20 to indicate the fragrance container 16 is empty. For example, the sensor assembly 20 may be a binary level indicator that outputs a first value, corresponding to “not empty” when the level of fragrance liquid in the container 16 is above a threshold level, and outputs a second value, corresponding to “empty” when the level of fragrance liquid in the container 16 is lower than the threshold level. This is diagrammatically shown only in FIG. 3, where the threshold is indicated by the horizontal level of the Section S-S line, and the indicated level labeled “Not empty” is above this threshold while the indicated level labeled “Empty” is below this threshold. The threshold is preferably chosen to be relatively close to the bottom of the fragrance container 16, as shown in FIG. 3, so that the “Empty” indication is triggered when the liquid level is close to the bottom of the container 16 (that is, the liquid is almost gone); however, the threshold is some distance above the very bottom of the container 16 so that there is still some fragrance liquid remaining in the container 16 when the “Empty” indication is triggered, thereby providing the user with some time cushion to replace or refill the fragrance container 16 before the air freshener 10 stops delivering the airborne fragrance output due to completely running out of fragrance liquid. In some embodiments, hysteresis is incorporated into the level indicator output algorithm. In one approach, different thresholds are used for “Empty” and “Not empty” to account for the effect of surface tension at the liquid/air interface.

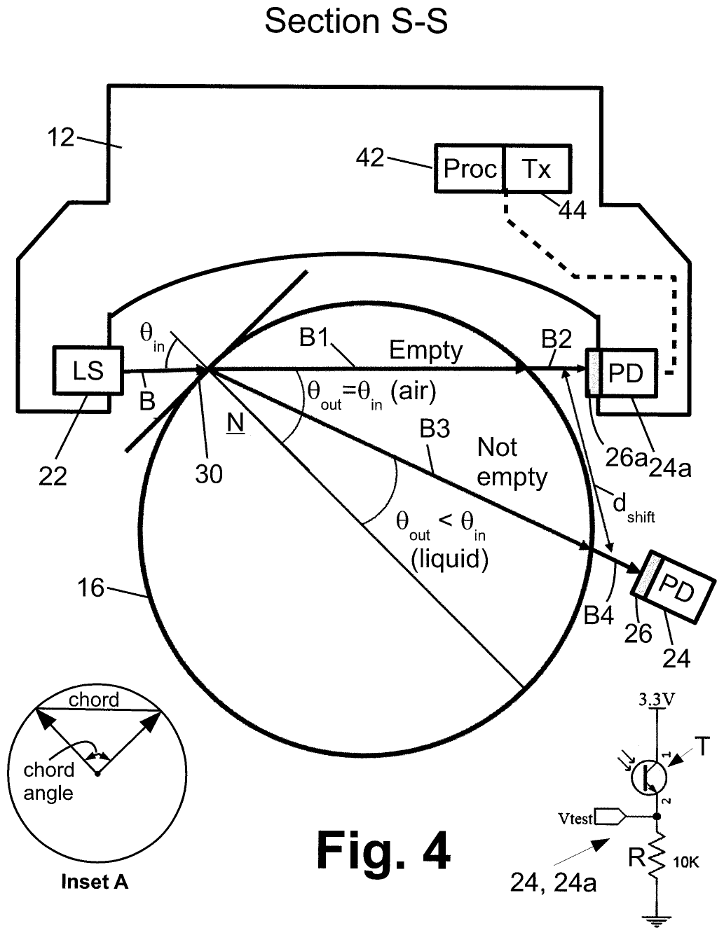

The sensor assembly 20 can take various forms. In general, the sensor assembly 20 includes a source 22 and a detector 24 (labeled only in FIG. 3; see also FIG. 4). In some preferred embodiments, the sensor assembly 20 employs optical sensing in which a color, time of flight, or intensity of light differs when the light is passing through the fragrance liquid (such that the container 16 is not empty) compared with when the light is passing through air (such that the container is empty). However, existing sensors of this type have some substantial difficulties when applied for detecting whether the fragrance container 16 is empty. One problem relates to sensitivity and robustness. The fragrance container 16 is usually a consumable item (replaced rather than refilled), and is desired to be an inexpensive disposable item. As such, manufacturing tolerances for the fragrance container 16 are usually not strict, and there can be wide item-to-item variation in characteristics of the fragrance container 16 such as its roundness (assuming it is designed to have a circular cross-section as in the embodiment of FIGS. 1-3), its precise placement when installed via the connector 14, the smoothness and precise contouring of its walls, and so forth. Because of this, the optically sensed color, time of flight, or other characteristics can vary significantly from one container to another.

A further problem is that the sensor assembly 20 can interfere with the installation (and subsequent removal/replacement) of the fragrance container 16. The sensor assembly 20 is suitably placed near the bottom of the container 16 in order to operate at an empty/not empty threshold level that is near the bottom of the container 16. But the user typically grips the container 16 near its bottom when screwing it into or out of the (e.g. threaded or bayonet) connection 14.

With continuing reference to FIGS. 1-3 and with further reference to FIG. 4, these difficulties are addressed in an illustrative sensor assembly 20 which leverages the different refraction angle at a container/air boundary compared with a container/fragrance fluid boundary. In this sensor assembly 20, the source 22 comprises a laser diode, light emitting diode (LED) with focusing lens, or other light source (LS) 22 that outputs a light beam B, and the detector 24 comprises a photodetector 24 with optional spectral filter 26. For example, the filter 26 can be designed to remove stray light and/or may be a bandpass filter that preferentially passes a center wavelength of light emitted by the light source 24. The light source 24 is disposed on or in the electrically powered fragrance dispenser 12 and is positioned to transmit the light beam B into the fragrance container 16 at a non-normal angle of incidence θᵢₙ respective to a wall 30 of the fragrance container 16 upon which the light beam B impinges. The angle of incidence θᵢₙ is measured off the surface normal, denoted as N in FIG. 4. According to Snell's law, the output angle, Nut of the light beam inside the container 16 after passing through the wall 30 is given by:

n₁ sin(θᵢₙ)=n₂ sin(θₒᵤₜ) (1)

Outside the container 16 the ambient medium is air, having refractive index n₁=1.00. Inside the container 16, the medium into which the light beam enters is either air (if the container is empty, by which it is meant that the liquid level is below the threshold indicated by Section S-S in FIG. 3) or the medium is the fragrance liquid. If the medium into which the light beam B enters is air, having n₁=1.00, then θₒᵤₜ=θᵢₙ and as seen in FIG. 4 the beam B1 inside the container 16 continues in the same direction after passing through the wall 30 as the beam B was traveling before passing through the wall 30. Likewise, at the opposite wall when the beam B1 passes back outside of the container 16 as beam B2, it remains traveling in the same original direction θᵢₙ. The photodetector 22 is disposed on or in the electrically powered fragrance dispenser 12 and at a position which is not in the path of the light beam B2 after passing through the fragrance container 16 when the light beam B1 is not refracted by fragrance liquid in the fragrance container 16.

On the other hand, if the medium into which the light beam enters is the fragrance liquid (because the container is not empty, and the liquid level is above the threshold indicated by Section S-S in FIG. 3), then the refractive index n₂ of the medium inside the container is greater than 1.00. By way of non-limiting illustrative example, Dowanol™ hydrophilic glycol ether fluid has n₂˜1.41. Using these values, and assuming θᵢₙ=45° (as in the illustrative example of FIG. 4), solving Equation (1) for θₒᵤₜ yields θₒᵤₜ=30.1°. In general, since the liquid virtually always has a larger refractive index than air (n₂>n₁), it follows from Equation (1) that θₒᵤₜ<θᵢₙ and the light beam B3 passing through the container 16 is bent toward the surface normal N. For these values, and assuming the container has a circular cross-section of diameter 30 mm, the light beam B3 (for liquid) passes out of the container 16 at a distance d_{shift=}8 mm away from where the beam B2 (for air) passes out. Because of this large shift, the photodetector 24 is in the path of the light beam after passing through the fragrance container 16 when the light beam B3 is refracted by fragrance liquid in the fragrance container 16.

Because of the large shift (dshift) thus obtained between empty (passing through air) and not empty (passing through fragrance liquid), the sensor assembly 20 of FIG. 4 is robust against typical item-to-item variations in the fragrance container 16. So long as these changes are not so large that the “straight-through” beam sequence B→B1→B2 hits the photodetector 24, the sensor should provide accurate indication of empty or not empty. Moreover, this “straight-though” beam is not affected by variations in the tilt of the wall 30 of the fragrance container 16 upon which the light beam B impinges (since θₒᵤₜ=θᵢₙ regardless of the tilt of the wall 30) and is likewise insensitive to the tilt of the wall at the opposite side (at the beam B1 beam B2 transition). The tilt of the wall 30 will affect the exact angle θₒᵤₜ of the beam B3 in the “not empty” condition, but so long as the beam width is large enough to hit the photodetector 24 even with tilt variation, the sensor assembly will work properly.

In an alternative embodiment, the photodetector 24 (and optional filter 26) arranged to detect the refracted beam B4 in the “not empty” case is replaced by a photodetector 24a (and optional filter 26a) that is arranged to detect the “through” beam B2 in the “empty” case. An advantage of this design is that the sensor assembly 20 (including source 22 and photodetector 24a) can be positioned nearer to an edge of the fragrance container 16. This is shown in FIG. 4 by the position of the dispenser 12 so that the arms are positioned to mount photodetector 24a. By contrast, when using the position of photodetector 24 to detect the refracted beam B4, the dispenser 12 would need to be rotated around and have longer arms to mount both the source 22 and detector 24 (not shown in FIG. 4).

The light source 22 is positioned such that the path of the light beam passing through a circular cross-section of the fragrance container 16 when the light beam is not refracted by fragrance liquid in the fragrance container 16 (that is, the beam B1 shown in. FIG. 4) defines a chord of the circular cross-section. Similarly, the path of the light beam passing through the circular cross-section of the liquid container 16 when the light beam is refracted by liquid in the container 16 (that is, the beam B3 shown in FIG. 4) defines another (i.e. refracted) chord of the circular cross-section. If the sensor assembly 20 is placed near the edge of the container, then this chord will have a small angle. (As diagrammed in “Inset A” of FIG. 4, the angle of a chord of a circle is the arc of the circle that is subtended by the chord. The term “chord angle” is also used herein to denote this angle of the chord. The chord can also be similarly defined in the context of a cross-section that is not perfectly circular, e.g. which is oval or has one or more indentations or protrusions and/or so forth). If the chord angle is small, then the light source 22 and photodetector 24 or 24a are both positioned near an edge of the container 16, and hence will minimally interfere with user access to the container 16 when installing or removing the container 16 from the connector 14. This is most easily achieved in embodiments employing the photodetector 24a since the refraction producing refracted beam B3 drives the beam deeper “into” the container. In some embodiments, the light source 22 is positioned such that the path of the light beam passing through a circular cross-section of the fragrance container when the light beam is not refracted by fragrance liquid in the fragrance container (that is, beam B1) defines a chord of the circular cross-section in which the angle of the chord is less than or equal to 100 degrees. In some even more advantageous embodiments, the angle of the chord is less than or equal to 55 degrees. In the illustrative example of FIG. 4, the angle of the chord is 45 degrees.

In an illustrative example shown in the lower right inset of FIG. 4, in this illustrative embodiment the photodetector 24 (or, in the alternative embodiment, the photodetector 24a) is a phototransistor (T) that is connected to +3.3V via a pull up resistor (R), which is a 10 k-ohm resistor in the illustrative example. The output (Vtest) has a low voltage when the phototransistor (T) is illuminated by light (because the phototransistor “shorts” the line to ground), and high voltage when the phototransistor (T) is not illuminated by light (the phototransistor remains open). This is merely an illustrative example, and other designs for the photodetector 24 (or photodetector 24a) are contemplated, e.g. using a photodiode, phototransistor, or other photonic device in a suitable biasing circuit topology.

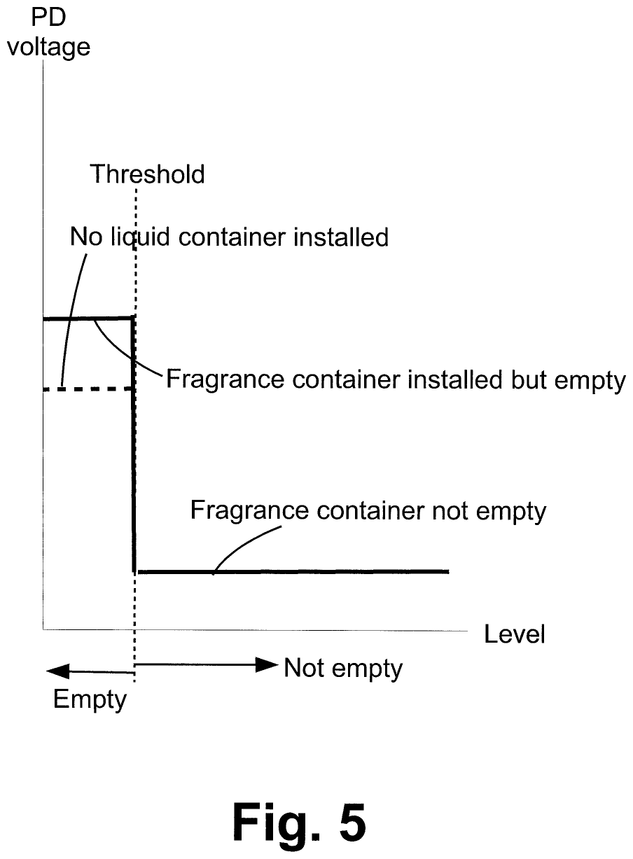

With reference now to FIG. 5, the photodiode voltage measured by the photodetector 24 of the embodiment of FIG. 4 is shown as a function of liquid level in the fragrance container 16. As seen, at the empty/not empty threshold (corresponding to the horizontal level of the Section S-S in FIG. 3), the voltage transitions from a low voltage value for liquid level above the threshold (because here the beam is following the refracted beam B3 of FIG. 4 so that the photodetector 24 is in the path of the refracted beam B3 and is illuminated by the light beam B4) to a high voltage value for liquid level below the threshold (because here the beam is following the not-refracted path of beam B1→B2 of FIG. 4 so that the photodetector 24 is not in the path and is not illuminated by the light beam B2).

If using the photodiode 24a positioned in the unrefracted light path B1→B2 of FIG. 4, and the transistor-based detector of FIG. 4, lower right inset is used, then the voltages will be reversed, i.e. when the container is empty the light beam B2 illuminates photodetector 24a producing a low voltage; whereas when the container is not empty the refracted light beam B4 does not illuminate the photodetector 24a leading to a high voltage.

A further advantage of the sensor assembly of FIG. 4 is that, depending upon the light absorption of the empty container 16, it may be possible to distinguish between the cases of (1) the air freshener 10 having an installed but empty container 16, and (2) the air freshener 10 not having any container installed at all. As diagrammatically shown in FIG. 5 for the case of using the photodetector 24 positioned to detect the refracted beam, in the latter case (no container installed) the output of the photodetector 24 is lower, indicating the light beam is detected at a beam intensity that is greater than a threshold value corresponding to the fragrance container not being installed. More generally, an indication that liquid container is not installed in the fluid dispenser is based on the light beam being detected at an intermediate value between being detected to pass through the chord of the cross-section of the liquid container and being not detected to pass through the chord of the cross-section of the liquid container. This can be particularly advantageous in the case of air fresheners installed in a commercial location where fragrance containers may be removed by vandals or thieves.

With reference back to FIGS. 3 and 4, if the sensor assembly 20 detects the fragrance container 16 is empty then this can be indicated in various ways. In one approach (shown in FIG. 3), an LED indicator 40 is provided on the electrically powered fragrance dispenser, which is lighted if the sensor assembly 20 detects the fragrance container 16 is empty. The LED 40 may be suitably labeled, e.g. by text indicating “Refill” as shown in FIG. 3. The LED 40 may for example be driven by an electronic processor 42 (e.g., a microprocessor or microcontroller) that is operatively connected to receive the output from the photodetector 24 and is programmed to control the LED 40.

The LED 40 or other indicator mounted on the air freshener 10 may provide a visual cue to the user indicating the fragrance container 16 need to be refilled or replaced (depending on design). However, the LED 40 will not be visible to the user if the air freshener is placed in an inconspicuous or hidden location, such as being plugged into an electrical outlet located behind a chair or other furniture, as is sometimes the case.

Accordingly, in some embodiments the output is instead (or additionally) provided to a mobile device (e.g. a cellular telephone, i.e. cellphone, tablet computer, or so forth) by way of wireless communication. To this end, as shown in FIG. 4, a transmitter or transceiver 44 is disposed on or in the electrically powered fragrance dispenser 12 and operatively connected to output a wireless signal indicating an output of the photodetector 24. The transmitter or transceiver 44 typically operates under control of the electronic processor 42. The transceiver or transmitter 44 may, for example, be a Bluetooth™ transmitter or transceiver, a WiFi transceiver, and/or so forth.

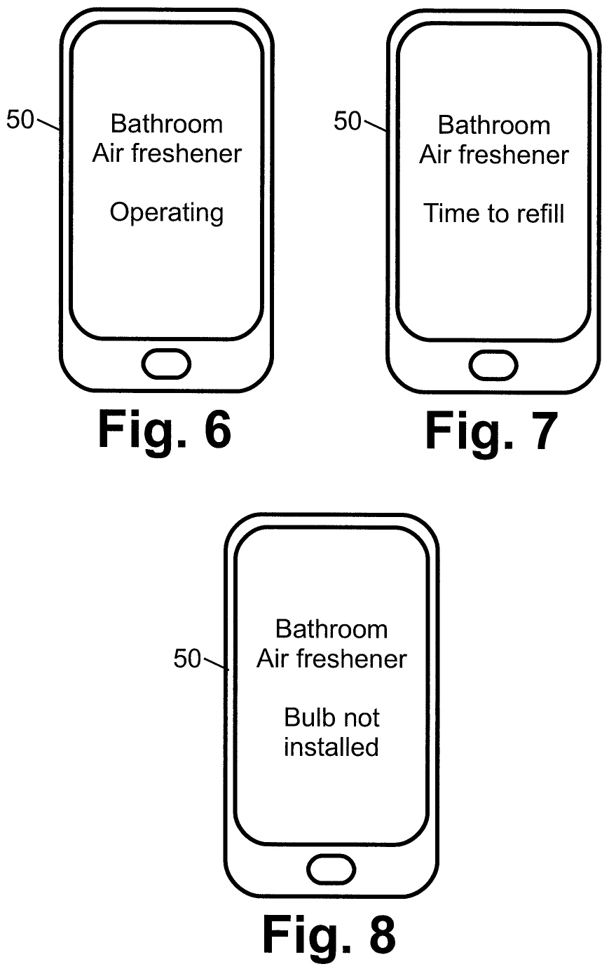

With continuing reference to FIG. 4 and with further reference to FIGS. 6-8, a mobile device 50 in the illustrative form of a brick cellphone is loaded with an application program (“app”) that when run on the mobile device 50 receives the wireless signal from the transmitter or transceiver 44 using a complementary Bluetooth™, WiFi, or other wireless radio of the mobile device 50, and presents a suitable informative display, such as “Bathroom Air Freshener operating” (FIG. 6, corresponding to the sensor assembly 20 indicating the container 16 is not empty), or “Bathroom Air Freshener—Time to refill” (FIG. 7, corresponding to the sensor assembly 20 indicating the container 16 is empty). In embodiments in which the sensor assembly 20 distinguishes between the container being empty versus not installed at all, the mobile device 50 running the app may if appropriate display “Bathroom Air Freshener—Bulb not installed” (FIG. 8, corresponding to the sensor assembly 20 indicating the fragrance container, here referred to as a “bulb”, is not installed). The designation “Bathroom Air Freshener” is suitably determined based on a device identification (device ID) code transmitted by the transmitter or transceiver 44 under control of the electronic processor 42, or by some other device identification mechanism. It will be appreciated that the specific messages presented in FIGS. 6-8 are non-limiting illustrative examples.

In the embodiment of FIGS. 1-5, the photodetector 24 is disposed on or in the electrically powered fragrance dispenser 10 at a position which is in the path of the light beam after passing through the fragrance container when the light beam is not refracted by fragrance liquid in the fragrance container. As explained herein, this arrangement has certain advantages, e.g. the sensor assembly 20 can be positioned near an edge of the fragrance container 16. However, the opposite arrangement is alternatively contemplated, that is, an arrangement in which the photodetector is disposed on or in the electrically powered fragrance dispenser at a position which is in the path of the light beam after passing through the fragrance container when the light beam is refracted by fragrance liquid in the fragrance container.

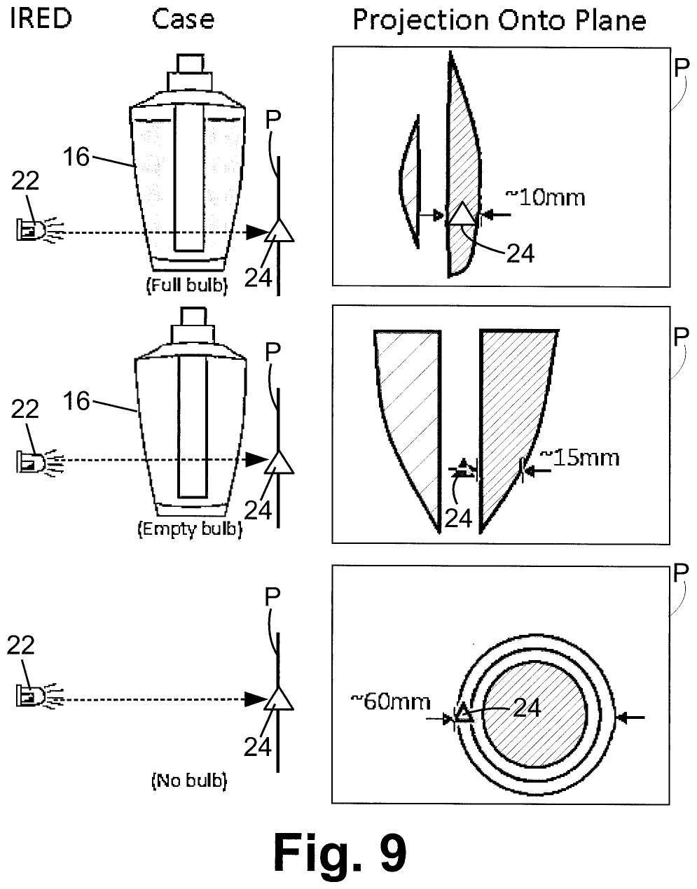

With reference now to FIG. 9, infrared mapping was performed to assess the impact of the fragrance container on the beam shape, using the arrangement employing the photodetector 24 arranged to detect the refracted beam B3→B4 and using the transistor based photodetector topology of FIG. 4, lower right inset. FIG. 9 shows three cases: the fragrance container 16 (i.e. bulb 16) is not empty (top drawing; labeled as a “full” bulb); the fragrance container 16 (i.e. bulb 16) is empty (middle drawing); and the fragrance container 16 (i.e. bulb 16) is missing (bottom drawing). The left side of each drawing illustrates the arrangement of the light source 22 and photodetector 24 relative to the bulb 16, along with an indication of a detection plane P at which an infrared detector array is placed. The right side of each drawing diagrammatically shows the infrared intensity distribution using shading (darker shading indicates higher infrared intensity). The location of the photodetector 24 in the plane P is also indicated in the right side diagram. In this illustrative experiment, the light source 22 is an infrared emitter, and the distance between the infrared emitter 22 and the plane P is 70 mm.

As particularly seen in comparing the middle drawing (empty bulb) versus the lower drawing (missing bulb), the presence of the bulb 16 significantly changes the distribution of light. In the case of the missing bulb (bottom drawing), the infrared distribution is radially symmetrical with highest intensity at the center, and a diameter of about 60 mm. By contrast, in the case of the empty bulb (middle drawing), the bulb horizontally breaks the beam up into two lobes separated by a low intensity central region, and also bends the two lobes upward. If the photodetector 24 is located in the low intensity central region between the two lobes, then a high sensor voltage is measured. Table 1 lists the sensor voltages measured by the photodetector 24 in each case. As can be seen, due to the bifurcated intensity distribution introduced by the bulb 16 coupled with placement of the photodetector 24 in the gap between the two lobes in the empty bulb case, a strong signal difference is seen between the empty bulb and missing bulb cases, thereby providing high discriminative capability between these two cases. In general, when the liquid container is not installed the detected light beam is usually expected to be at an intermediate value between being detected to pass through the chord of the cross-section of the liquid container and being not detected to pass through the chord of the cross-section of the liquid container (i.e. between the empty and not empty readings). Such ability to distinguish whether the bulb is empty or missing may be useful, for example, in a public location in which bulbs may be removed by vandals or the like.

TABLE 1

| Case | Sensor voltage |
|---|---|
| Non-empty bulb | 0.185 V |
| Empty bulb | 3.07 V |
| Missing bulb | 1.87 V |

The illustrative example of the sensor assembly 20 shown in FIGS. 1-4 has certain advantages, as discussed hereinabove. However, the sensor assembly 20 operates on the assumption that the air freshener is mounted or positioned in a fixed design-basis orientation. This will be achieved, for example, if the AC wall plug 18 shown in the example of FIG. 2 is plugged into a vertically oriented electric wall outlet. However, some air fresheners may be intended for use in different orientations. For example, an air freshener may be intended for use in a vehicle, or the electrically powered fragrance dispenser of the air freshener may have a swivel or gimbal mount via which the electrically powered fragrance dispenser can be rotated about at least one axis. In these cases, the assumption of a fixed design-basis orientation is no longer valid, and the level based sensor assembly of FIG. 4 may be inoperative and/or unreliable.

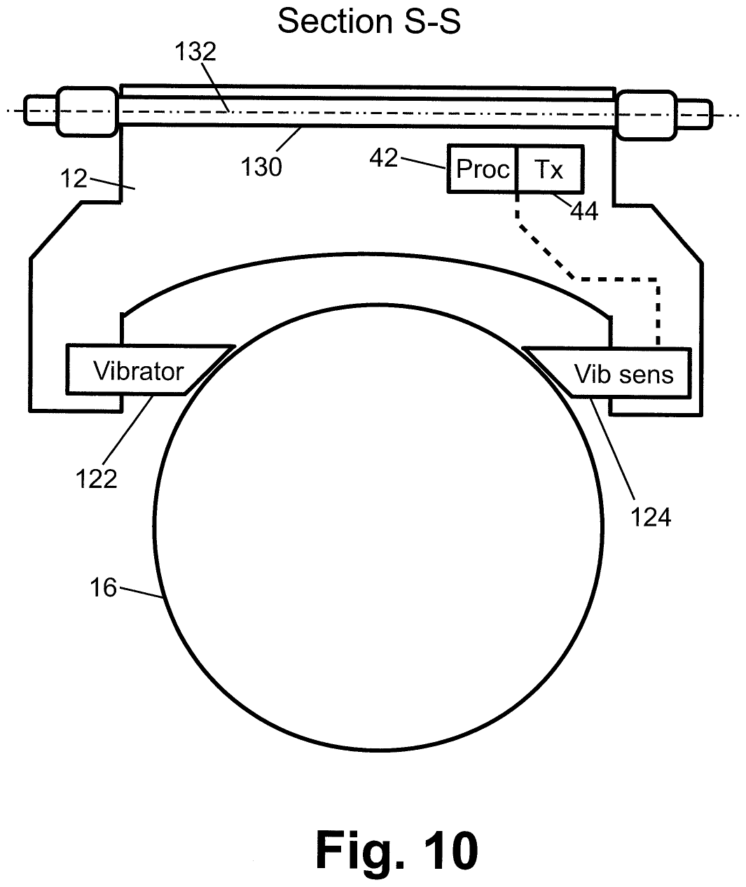

With reference now to FIG. 10, another embodiment of the sensor assembly 20 is shown, which does not rely upon the assumption of a fixed design-basis orientation. This air freshener is illustrated by way of the same Section S-S shown in FIG. 3; however, in this embodiment the source 22 of the sensor assembly comprises a vibrator 122 disposed on or in the electrically powered fragrance dispenser 12 and positioned to induce a vibration of the fragrance container 16. The detector 24 comprises a vibration sensor 124 disposed on or in the electrically powered fragrance dispenser 12 and positioned to detect the vibration of the fragrance container 16. The electronic processor 42 is programmed to determine whether the fragrance container 16 is empty of fragrance liquid based on a frequency and/or amplitude of the detected vibration of the fragrance container. The resulting output may be displayed via the LED 40 shown in FIG. 3 and/or ported off wirelessly by way of the wireless transmitter or transceiver 44 as already described. It may be noted that in this embodiment the container 16 does not need to be transparent or translucent as no light is passed into or out of the container.

The vibrator 122 can be in contact with the container 16, or can be at a standoff from the container 16, as long as the vibrator 122 can induce the vibration in the container 16. As an example of a vibrator 122 with a standoff, the vibrator 122 could be an ultrasonic transducer that transmits ultrasonic waves so as to induce the vibration of the fragrance container 16. In general, the induced vibration may be an impulse force or a steady state frequency. The vibration sensor 124 can be in contact with the container 16, or can be at a standoff from the container 16, as long as the vibration sensor 124 can detect the vibration of the container 16. As an example of a vibration sensor 124 with a standoff, the vibration sensor 124 could be an ultrasonic transducer that induces a voltage in response to the ultrasonic waves generated by the vibrating container 16. Advantageously, the vibrator 122 and vibration sensor 124 can be variously located, as there is no required position. Preferably, neither component is located on one of nodes of the vibrational mode shape of the vibrations induced by the vibrator 122.

In one embodiment, the vibration sensor 124 (and/or post-processing of the detected vibration by the processor 42) measures the frequency response of the container 16 to the induced vibration. The shift in natural frequency (from an impulse force), and/or the change in vibrational amplitude at a given excitation frequency, can be used as indicative of whether the container 16 is empty or not empty. This is due to the vibration amplitude and frequency being dependent upon mass and density characteristics of the container 16. The excitation frequency is preferably targeted at a frequency that provides a strong difference in amplitude between the empty and not empty states of the container 16. The natural frequency and/or vibrational amplitude of the empty versus not empty container can be determined empirically by measuring the vibrational response to various vibrator inputs, or can be computed using mass-and-spring constant modeling approaches using parameters such as the container mass, the mass density of the fragrance liquid, volumes, and so forth.

Advantageously, the vibrational response is expected to be substantially unaffected by orientation of the container 16. Hence, for example, the electrically powered fragrance dispenser 12 can have a swivel or gimbal mount (e.g. illustrative swivel mount 130 shown in FIG. 10) via which the electrically powered fragrance dispenser 12 (and hence the connected fragrance container 16) can be rotated about at least one axis 132, and the vibrational sensor assembly of FIG. 10 will operate to assess whether the container 16 is empty for a range of different rotational positions. Preferably, during empirical calibration of the vibrational sensor assembly, test measurements of the vibrational response are taken at different rotational positions spanning the expected range of positions during actual use, so as to ensure sufficient robustness of the sensing against such orientation variations. In some embodiments, the output of the vibration sensor 124 may be filtered or gated to remove vibrational signal components that are attributable to sudden movement of the container 16. Such filtering or gating may be particularly useful in the case of an air freshener to be used in an environment such as a vehicle in which sudden movements can be expected.

Similarly to the optically-based embodiment of FIG. 4, the vibration-based embodiment of FIG. 10 can also detect if the fragrance container 16 is missing. In this case, the vibrational response will typically be a null response, since the fragrance container 16 is the medium via which vibrations produced by the vibrator 122 are transmitted to the vibration sensor 124. Hence, if there is no fragrance container installed in the fragrance dispenser 12 then a null response will typically be detected, and this can be used to provide an indication that no container is installed, e.g. as described previously with reference to FIGS. 5, 8, and 9 (bottom drawing).

The illustrative embodiments have been directed to air fresheners. However, it will be appreciated that the disclosed approaches are more generally applicable to detecting whether a liquid container that is connected with a fluid dispenser is empty. To generalize, whether a liquid container connected with a fluid dispenser is empty is monitored as follows. The fluid dispenser is operated to dispense fluid by consuming liquid contained in the liquid container. In an air freshener this usually entails running the electrically powered fragrance dispenser 12. In the case of a hand sanitizer, the fluid dispenser may be a manual pump and the liquid is hand sanitizer liquid. Using the embodiment of FIG. 4, a light beam is directed by light source 22 through a chord of a circular cross-section of the liquid container (e.g., the chord along which the beam B1 of FIG. 4 travels), and the photodetector 24 detects whether the light beam passes through the chord of the circular cross-section of the liquid container. An indication that the fluid dispenser should be refilled is outputted if the light beam is detected to pass through the chord of the circular cross-section of the liquid container, e.g. via a visual indicator or a wireless transmitter or transceiver.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. An air freshener comprising:
an electrically powered fragrance dispenser having a connector configured to connect with a fragrance container which is transparent or translucent, wherein the electrically powered fragrance dispenser is operative to generate a fragrance output wherein the generation of the fragrance output consumes a fragrance liquid stored in the fragrance container;
a light source disposed on or in the electrically powered fragrance dispenser and positioned to transmit a light beam into the fragrance container at a non-normal angle of incidence respective to a wall of the fragrance container upon which the light beam impinges; and
a photodetector disposed on or in the electrically powered fragrance dispenser and at a position which is in the path of one of (i) the light beam after passing through the fragrance container when the light beam is not refracted by fragrance liquid in the fragrance container and (ii) the light beam after passing through the fragrance container when the light beam is refracted by fragrance liquid in the fragrance container.

2. The air freshener of claim 1 further comprising:
an indicator that provides an indication based on a signal output by the photodetector, the indication being one of:
an indication that the fragrance container is empty, or
an indication that the fragrance container is not empty.

3. The air freshener of claim 2 wherein the indication that the fragrance container is empty is provided by lighting a light emitting diode (LED) disposed on the electrically powered fragrance dispenser and the indication that the fragrance container is not empty is provided by not lighting the LED disposed on the electrically powered fragrance dispenser.

4. The air freshener of claim 1 further comprising:
a wireless transmitter or transceiver disposed on or in the electrically powered fragrance dispenser and operatively connected to output a wireless signal indicating an output of the photodetector.

5. The air freshener of claim 4 further comprising:
a mobile device comprising a cellular telephone (cellphone) or tablet computer, the mobile device having loaded thereon an application program (app) operative to cause the mobile device to wirelessly receive the wireless signal indicating the output of the photodetector and based on the received wireless signal to display one of:
an indication that the fragrance container is empty, or
an indication that the fragrance container is not empty.

6. The air freshener of claim 4 further comprising:
a mobile device comprising a cellular telephone (cellphone) or tablet computer, the mobile device having loaded thereon an application program (app) operative to cause the mobile device to wirelessly receive the wireless signal indicating the output of the photodetector and based on the received wireless signal to display one of:
an indication that the fragrance container is empty,
an indication that the fragrance container is not installed, or
an indication that the fragrance container is not empty.

7. The air freshener of claim 1 wherein the photodetector is disposed at a position which is in the path of the light beam after passing through the fragrance container when the light beam is refracted by fragrance liquid in the fragrance container.

8. The air freshener of claim 1 wherein the photodetector is disposed at a position which is in the path of the light beam after passing through the fragrance container when the light beam is not refracted by fragrance liquid in the fragrance container.

9. The air freshener of claim 8 wherein the light source is positioned such that the path of the light beam passing through a circular cross-section of the fragrance container when the light beam is not refracted by fragrance liquid in the fragrance container defines a chord of the circular cross-section wherein the angle of the chord is less than or equal to 100 degrees.

10. The air freshener of claim 8 wherein the light source is positioned such that the path of the light beam passing through a circular cross-section of the fragrance container when the light beam is not refracted by fragrance liquid in the fragrance container defines a chord of the circular cross-section wherein the angle of the chord is less than or equal to 55 degrees.

11. The air freshener of claim 1 wherein the electrically powered fragrance dispenser is operative to generate the fragrance output by one of thermal heating of the fragrance liquid, piezoelectric energizing of the fragrance liquid, propellent-driven output of the fragrance liquid, or nebulization of the fragrance liquid.

12. The air freshener of claim 1 wherein the connector of the electrically powered fragrance dispenser is a threaded connector whereby the fragrance container screws onto the electrically powered fragrance dispenser.

13. The air freshener of claim 1 further comprising a spectral filter disposed in front of the photodetector.

14. A method of monitoring whether a liquid container connected with a fluid dispenser is empty, the method comprising:
operating the fluid dispenser to dispense fluid by consuming liquid contained in the liquid container;
directing a light beam into the liquid container;
detecting whether the light beam passes through a chord of a cross-section of the liquid container; and
outputting, via a visual indicator or a wireless transmitter or transceiver, an indication that the fluid dispenser should be refilled or replaced if the light beam is one of (i) detected to pass through the chord of the cross-section of the liquid container or (ii) not detected to pass through the chord of the cross-section of the liquid container.

15. The method of claim 14 wherein:
the chord is along a path of the light beam when the light beam is refracted by liquid in the liquid container; and
the outputting comprises outputting an indication that the fluid dispenser should be refilled or replaced if the light beam is not detected to pass through the chord of the cross-section of the liquid container.

16. The method of claim 14 wherein:
the chord is along a path of the light beam when the light beam is not refracted by liquid in the liquid container; and
the outputting comprises outputting an indication that the fluid dispenser should be refilled or replaced if the light beam is detected to pass through the chord of the cross-section of the liquid container.

17. The method of claim 14 further comprising:
outputting, via the visual indicator or the wireless transmitter or transceiver, an indication that liquid container is not installed in the fluid dispenser based on the light beam being detected at an intermediate value between being detected to pass through the chord of the cross-section of the liquid container and being not detected to pass through the chord of the cross-section of the liquid container.

18. The method of claim 14 wherein the liquid container is a fragrance container of an air freshener further comprising an electrically powered fragrance dispenser.

19. An air freshener comprising:
an electrically powered fragrance dispenser having a connector configured to connect with a fragrance container which is transparent or translucent, wherein the electrically powered fragrance dispenser is operative to generate a fragrance output wherein the generation of the fragrance output consumes a fragrance liquid stored in the fragrance container;
a vibrator disposed on or in the electrically powered fragrance dispenser and positioned to induce a vibration of the fragrance container;
a vibration sensor disposed on or in the electrically powered fragrance dispenser and positioned to detect the vibration of the fragrance container; and
an electronic processor programmed to determine whether the fragrance container is empty of fragrance liquid based on a frequency and/or amplitude of the detected vibration of the fragrance container.

20. The air freshener of claim 19 wherein the electrically powered fragrance dispenser has a swivel or gimbal mount via which the electrically powered fragrance dispenser can be rotated about at least one axis.

21. The air freshener of claim 19 further comprising:
a wireless transmitter or transceiver disposed on or in the electrically powered fragrance dispenser and operatively connected to output one of:
a wireless signal indicating the fragrance container is empty responsive to the electronic processor determining the fragrance container is empty of fragrance liquid, or
a wireless signal indicating the fragrance container is not empty responsive to electronic processor determining the fragrance container is not empty of fragrance liquid.

22. The air freshener of claim 21 wherein the wireless transmitter or transceiver is operatively connected to further output a wireless signal indicating the fragrance container is not installed in the fragrance dispenser responsive to electronic processor determining the vibration sensor detects a vibrational response which is a null response.

23. The air freshener of claim 21 further comprising:
a mobile device comprising a cellular telephone (cellphone) or tablet computer, the mobile device having loaded thereon an application program (app) operative to cause the mobile device to wirelessly receive the wireless signal output by the wireless transmitter or transceiver and to display the indication that is indicated by the received wireless signal.

24. The air freshener of claim 19 wherein the electrically powered fragrance dispenser is operative to generate the fragrance output by one of thermal heating of the fragrance liquid, piezoelectric energizing of the fragrance liquid, propellent-driven output of the fragrance liquid, or nebulization of the fragrance liquid.

25. The air freshener of claim 19 wherein the connector of the electrically powered fragrance dispenser is a threaded connector whereby the fragrance container screws onto the electrically powered fragrance dispenser.
